(19) United States
(12) Patent Application Publication
(10) Pub. No.: US 2025/0194932 A1
(43) Pub. Date: Jun. 19, 2025

(54) NON-INVASIVE MECONIUM AND BLOOD SCREENING DEVICE IN AMNIOTIC FLUID DURING THE BIRTH PROCESS

(71) Applicant: ATATURK UNIVERSITESI REKTÖRLUGU BILIMSEL ARASTIRMA PROJELERI ( BAP ) KOORDINASYON BIRIMI, Erzurum (TR)

(72) Inventors: AYLA KANBUR, Erzurum (TR); SERAP EJDER APAY, Erzurum (TR); BÜLENT ÇAVUSOGLU, Erzurum (TR); MIRAÇ DILRUBA GEYIKOGLU, Erzurum (TR); ÖMER ÇOBAN, Erzurum (TR)

(73) Assignee: ATATURK UNIVERSITESI REKTÖRLUGU BILIMSEL ARASTIRMA PROJELERI ( BAP ) KOORDINASYON BIRIMI, Erzurum (TR)

(21) Appl. No.: 18/692,843

(22) PCT Filed: Sep. 6, 2023
(86) PCT No.: PCT/TR2023/050916

§ 371 Date: Mar. 18, 2024

(30) Foreign Application Priority Data
Sep. 9, 2022 (TR) 2022/014016

(51) Int. Cl. A61B 5/00; A61B 5/145; A61B 5/1455

(52) U.S. Cl. CPC A61B 5/0082; A61B 5/0075; A61B 5/14507; A61B 5/1455; A61B 5/4362

(57) ABSTRACT

The present invention relates to the field of health. In particular, it is used in the fields of midwifery, obstetrics and gynecology, perinatology. During the birth process, it will be determined whether the fetus has meconium in the amniotic fluid surrounding the fetus and whether there is bleeding into the amniotic fluid.

### FIELD OF THE INVENTION

The present invention relates to the field of health. In particular, it is used in the fields of midwifery, obstetrics and gynecology, perinatology. During the birth process, it will be determined whether the fetus has meconium in the amniotic fluid surrounding the fetus and whether there is bleeding into the amniotic fluid.

### STATE OF THE ART

Meconium is the first product of the neonatal intestinal tract consisting of intestinal secretions, mucosal epithelial cells and solid elements of amniotic fluid, 85-95% of which is water. Depending on the amount of meconium, meconium-stained amniotic fluid can vary from light green, dark green and viscous to dense with particles.

Meconium, which begins to be produced in fetal life at 10-12 weeks, is rarely seen in amniotic fluid before 34 weeks of gestation. Meconium passage does not occur before 34 weeks of gestation because the intestines do not have strong peristaltic movements and the anal sphincter is in tonic contraction. However, as the gestational week progresses, neuronal transmission increases, the parasympathetic system matures and peristalsis becomes stronger, and the ‘motilin’ hormone that provides peristalsis in the fetus increases in direct proportion to the gestational age, meconium passage in the fetus begins to be seen.

Meconium-stained amniotic fluid occurs in 5-24% (mean 13%) of normal pregnancies (5.1% in preterm, 16.5% in term, 27.1% in postterm) and is a possible indicator of fetal distress. Perinatal mortality is 3-22.2% and neonatal mortality is 7-50% in infants with meconium in amniotic fluid. In intrauterine life, meconium may be discharged into the amniotic fluid under certain circumstances (due to compression of the fetal head and/or umbilical cord, due to fetal hypoxia, etc.). Passage of meconium into the amniotic fluid causes fetal distress. In this situation, hypoxemia, acidosis and asphyxia may develop in the fetus. Respiratory distress develops in 20-33% of newborns with meconium-stained amniotic fluid and meconium aspiration syndrome (MAS) develops in 2-10%. Meconium aspiration is an inflammatory condition caused by contamination of amniotic fluid with meconium and its penetration into the lungs. It is one of the leading causes of respiratory distress leading to the need for intensive care in newborns. It can also lead to pulmonary and neurodevelopmental sequelae both in the short and long term. When we look at the prognosis of the disease, it is seen that there are short-term and long-term complications and morbidity/mortality rates are high. Short-term complications of the disease include air leaks, bacterial pneumonia, sepsis and persistent pulmonary hypertension. Although there is currently insufficient information about the long-term neurodevelopmental prognosis of patients with meconium aspiration syndrome, it is known that the incidence of cerebral palsy, convulsions and mental retardation is higher compared to the normal population. It has also been reported that approximately 20% of these patients have neurodevelopmental retardation.

Another risky situation that can occur during labor is bleeding. During this period, 80% of bleeding occurs due to abruption of placenta, 10% due to placenta previa and 10% due to other causes. Placenta previa is the placement of the placenta in the lower segment of the uterus. Abruption of the placenta is premature separation of the placenta. Bleeding during labor is an important cause of maternal and neonatal morbidity and mortality. Bleeding during this process is among the most important causes of intrapartum death. Placenta previa and abruption of placenta can cause serious conditions such as bleeding, hypovolemic shock, postpartum hemorrhage, acute renal failure, disseminated intravascular coagulation disorder, thrombophlebitis and sepsis, leading to maternal mortality. In addition, intrauterine fetal deaths occur due to intrauterine hypoxia as a result of bleeding. Bleeding into the amniotic fluid is difficult to detect. Ultrasonography is less reliable for bleeding into the amniotic fluid.

Serious fetal complications can occur if the amniotic fluid contains meconium and there is bleeding into the amniotic fluid. Therefore, early diagnosis of the presence of blood in the amniotic fluid and the presence of meconium in the amniotic fluid during the birth process is important. The presence of blood and meconium in the amniotic fluid can be seen during the birth process when the amniotic membranes open and the amniotic fluid is discharged. The amniotic membrane usually opens at the end of the first stage of labor. This is a late stage for monitoring the amniotic fluid for blood and meconium.

To date, many studies have been carried out to diagnose meconium amniotic fluid. Ultrasound scanning has been used to study the density of amniotic fluid. This is a scan for echogenic liquor in the amniotic fluid (turbidity and density of the amniotic fluid). Echogenic areas in the amniotic fluid are scanned by ultrasound and emphasized to be associated with meconium. However, studies have shown that the echogenic liquor seen on ultrasound scan is not always meconium. Studies have found that echogenic areas are more commonly associated with the vernix caseosa (white cream-like substance that covers the fetal body) found in amniotic fluid. Therefore, ultrasound is not a reliable screening method for amniotic fluid with meconium.

The diagnosis of amniotic fluid with meconium can also be made when meconium is detected in the amniotic fluid obtained by amniocentesis performed when necessary in the later weeks of pregnancy. However, amniocentesis is not a preferred method for meconium diagnosis because it is an invasive procedure and carries many complication risks (bleeding, fetal injury, premature rupture of membranes, premature labor, etc.). It is only used to screen for genetic diseases or to determine fetal maturity.

Artificial opening of the amniotic membrane in the early stages of labor (amniotomy) for the early diagnosis of meconium amniotic fluid is not a routinely recommended practice. Premature opening of the membranes can lead to maternal and fetal infections (chorioamnionitis), umbilical cord prolapse and fetal hypoxia and asphyxia due to umbilical cord compression.

There are no devices on the market for this purpose.

Some examples of patents were found in national and international patent and model searches. But they are not in widespread use and these examples do not have the same characteristics as our invention. Some of the examples are listed below.

Patent No: U.S. Pat. No. 7,515,948 B1 PHOTOACOUSTIC ANALYZER OF REGION OF INTEREST IN A HUMAN BODY (Apr. 7, 2009) [Inventors: Michal Balberg, Jerusalem (IL); Revital Pery Shechter, Rishon LeZion (IL); Michal Olshansky, Tel Aviv (IL)]

Region of interest (ROI) is also defined in this patent. It was stated that at least one characteristic of the region of interest could be monitored non-invasively (including oxygen saturation and amniotic fluid content). In this patent, it is envisaged that a photoacoustic effect (photoacoustic can be defined as vibration caused by light) can be created when the region of interest is illuminated with pulsed light, and the acoustic effect created can be collected and processed by acoustic sensors. Thus, it was stated that information about the characteristics of the region of interest would be obtained.

In this patent, the probe has acoustic sensors and a fiber positioned to send pulsed light into the tissue. Light is sent through this fiber and an acoustic effect is created. The acoustic effect is collected by acoustic sensors and the data is processed to collect information about the tissue. It was also stated that the fiber can be used separately from the sensors (proposed positioning for meconium analysis). Here, the environment was stimulated with pulsed light and the photoacoustic effect was collected with acoustic data collectors.

The method used in this patent is completely different from the method proposed by us. This patent aims to measure several different parameters and focuses on oxygen saturation. It is planned to obtain data using photoacoustic method. In our invention, color reflectance measurement will be used and only amniotic fluid color examination will be performed.

Patent No: U.S. Pat. No. 8,644,900 B2 METHOD AND APPARATUS FOR NONINVASIVELY MONITORING PARAMETERS OF A REGION OF INTEREST IN A HUMAN BODY (Feb. 4, 2014) [Inventors: Michal Balberg, Jerusalem (IL); Revital Pery Shechter, Rishon LeZion (IL); Michal Olshansky, Tel Aviv (IL)]

In the above-mentioned patent, the “region of interest (ROI)” is defined and it is claimed that many parameters such as maternal tissues, fetus, amniotic fluid, oxygen saturation can be analyzed with the proposed method. In this patent, the region of interest may be different places. For example, fetal blood vessels can be examined for oxygen saturation or abdominal and pelvic cavities for pleural, pericardial, peritoneal examinations, etc.

The proposed method uses an acoustic (ultrasound) unit, light source, detector and control unit. The method proposed in the patent is as follows. The light from the light source passes through the area to be examined and reaches the detector. (The detector and light source are positioned so that the detector can collect the emitted light). With ultrasonic welding, the area to be examined in the region to be examined is stimulated. Since light will spread over a very large area, ultrasonic stimulation of the area to be examined causes the light passing through that area to be modulated at the frequency of the ultrasonic source. This is described in the patent as ultrasonically labeled light. Thus, it was mentioned that labeled light can be easily distinguished from unlabeled light. In this method, light is emitted from the source, propagates through the region and reaches the detector. In this method, information is gathered by the light emitted in the environment rather than reflected light. It is also supported by an acoustic unit.

It was emphasized that the light source used could be a narrow bandwidth laser diode and that there should be at least two, one at 605-805 nm and the other at 800-1300 nm. It was emphasized that Photomultiplier tube, image pixel array, photodiode can be used as detectors.

The method used in this patent is different from the method proposed by us. In this patent, maternal and fetal tissues are examined ultrasonically. In this patent, the product is said to work by the method of obtaining ultrasonic images by the propagation of light supported by ultrasonic waves in different tissues of the mother and fetus. This patent is a continuation of the previously mentioned patent (Patent No: U.S. Pat. No. 7,515,948 B1 PHOTOACOUSTIC ANALYZER OF REGION OF INTEREST IN A HUMAN BODY) and was obtained by adding light to the method. Therefore, it does not have the same characteristics and does not have the same purpose as the product recommended by us. In addition, in our invention, we will use a source with a broad spectrum between 400 nm and 1100 nm as a light source and a CCD detector as a detector. We also propose a color reflection method rather than ambient diffuse light. And we will only examine the color reflection in the amniotic fluid. We will not examine any fetal or maternal tissues.

Patent No: WO92/00699 A1 Meconium Monitoring System (23.01.1992) [Eric S. G. Genevier, London; Philip J. Steer, Kingdyon upon Thames; Peter J. Danielian, Aberdeen; Nigel J. Randall, London; Robin W. Smith, Crovdon, all of England]

U.S. Pat. No. 5,361,759 Meconium Monitoring System (Nov. 8, 1994) [Eric S. G. Genevier, London; Philip J. Steer, Kingdyon upon Thames; Peter J. Danielian, Aberdeen; Nigel J. Randall, London; Robin W. Smith, Crovdon, all of England]

A system for in vivo monitoring of the presence and concentration of meconium or blood in amniotic fluid during labor by spectral analysis.

It comprises a probe to insert into the uterus. This probe has a flexible body that houses an optical cell. The probe has a small opening for amniotic fluid to enter the cell. A fiber optic cable connects the cell to a light source and a spectral analyzer, and the probe comprises a structure to protect the fiber optic cable from any light scattered by the wall of the uterus or fetus.

While this method deals with the spectral analysis of reflected light and tries to generate information spectrally based on the wavelength and intensity of the reflected light, our proposed method will provide information about the color of the amniotic fluid from the reflected light. Although there is a similarity in terms of reflected light, we are basically based on “color measurement”.

In addition, this patent requires the amniotic fluid to pass through the designed probe for measurement. There is an opening in the probe (which means that fluid is removed from the sac). Therefore, it is an invasive method. It is proposed to measure the amniotic fluid as it passes through the device and collect spectral information.

In the method of the present invention, the color of the amniotic fluid will be determined by the reflection method without fluid intake from the sac. We propose a non-invasive method.

Patent No: US 2010/0324391 A1 Device and Method for Identification of Meconium in Amniotic Fluid [Genady Kostenich, Bat Yam (IL); Sol Kimel, Haifa (IL); Arie Orenstein, Tel Aviv (IL); Reuben Achiron, Tel Aviv (IL); Eliahu Pewzner, Modiin Ilit (IL) (Dec. 23, 2010)]

Methods and devices including a long probe for in vivo detection of meconium in amniotic fluid retained in an amniotic sac based on the detection of the presence of zinc-coproporphyrin I (ZnCP) are described. ZnCP is excited at about 405 nm and emits characteristic fluorescence centered at about 580 nm and less intense at 630 nm. If meconium is present in the amniotic fluid, the zinc coproporphyrin I (ZnCP) component of meconium absorbs the excitation light (405 nm) and fluoresces to emit light with a characteristic wavelength, e.g. at 580 nm and/or 630 nm.

This method is completely different from the method we propose. While our proposed method is based on the detection of reflected light and provides information about amniotic fluid from color determination, this method is based on the collection of light emitted by the excitation of the ZnCP component in the presence of meconium (which is 580 nm). Also the light source used is a 405 nm laser source.

U.S. Pat. No. 6,044,284 Apparatus and method for measuring the concentration of meconium in amniotic fluid (28 Mart 2000) (Eisenfeld et al.)

The present invention is directed to an apparatus and method for measuring meconium concentrations in amniotic fluid. In this method, several sensors are placed in different compartments of the fetus in the amniotic sac to obtain results.

U.S. Pat. No. 5,713,351 Intrauterine meconium detection system (3 Feb. 1998) (Billings et al.)

It is a method used to enter the uterus by piercing the amniotic sac with the help of a probe, to draw amniotic fluid into an observation unit along the probe lumen and to determine whether the amniotic fluid contains meconium or blood. In this model, again, invasive sampling of amniotic fluid was performed. It is seen that the integrity of the amniotic membrane is disrupted and the probe is delivered to the baby in the womb.

U.S. Pat. No. 5,172,693 Prenatal non-invasive detection of meconium stained amniotic fluid (22 Aralik 1992) (Michael C. Doody)

This method attempts to detect meconium non-invasively in amniotic fluid based on the detection of the fluorescence of the bilirubin component of meconium in meconium-stained amniotic fluid. The starting point is the presence of bilirubin in meconium. If bilirubin is detected in the amniotic fluid, this can be used as an indicator of meconium. A probe is inserted into the body (preferably transvaginally) and operates by reflecting monochromatic excitation light (produced by an argon laser) with a wavelength of 488 nm or 514 nm from the probe through the body tissue into the amniotic sac. In this model, only bilirubin screening is performed and a transvaginal catheter is also inserted.

Patent No: DE212012000011U1 Non-invasive detection of meconium in the amniotic fluid (25 Apr. 2013) The invention provides a system and method for detecting meconium in amniotic fluid released in pregnant women, including a collection body, such as a sanitary pad having a meconium detector.

The present invention is primarily intended for use by a woman working at home. In this model, collection of amniotic fluid with the pad is essential.

The models and inventions seen in the literature have generally not only focused on the examination of amniotic fluid, but have also evaluated different parameters in various tissues. Many of these patents are evaluated by an invasive procedure. Usually, amniotic fluid is sampled to assess the presence of meconium and blood.

The fact that our invention does not damage the amniotic membrane, does not require taking an amniotic fluid sample, is portable and convenient, non-invasive, does not require any probe, cable, sample collection for evaluation, and can quickly evaluate the result shows its importance and superiority. The product has potential for commercialization. The portability of the product increases its potential for commercialization. Since birth is a continuous action, the product to be developed is likely to have a wide range of uses.

### DEFINITION OF THE INVENTION

Said invention eliminates the disadvantages described in the state of the art and fulfils the needs.

The present invention relates to the field of health. In particular, it is used in the fields of midwifery, obstetrics and gynecology, perinatology. During the birth process, it will be determined whether the fetus has meconium in the amniotic fluid surrounding the fetus and whether there is bleeding into the amniotic fluid.

A device is needed to assess the amniotic fluid for blood and meconium during labor. The aim of the present invention is to develop a device for the screening of amniotic fluid. Thus, it is planned to contribute to the reduction of fetal and neonatal deaths due to causes that can be prevented by early diagnosis.

Diagnosing meconium and bloody amniotic fluid early in the birth process, reducing neonatal interventions due to these problems, reducing the need for neonatal intensive care, reducing neonatal intensive care unit admissions, increasing maternal health, reducing the loss of time and labor of healthcare personnel are other aims of the present invention.

The invention will reduce fetal and neonatal mortality due to the presence of blood and meconium in the amniotic fluid during labor. Early screening of amniotic fluid in this respect will provide early diagnosis and enable necessary precautions and interventions to be taken. It will also meet the need in this field since there is no method or device used for this purpose today.

Our invention works on the basis of reflection measurement. With this reflection, it can measure color changes in the amniotic fluid. Within the scope of the invention,

RGB led and tungsten halogen lamp will be used as light sources. While tungsten halogen gives light in all visible region wavelengths, with RGB led, all visible region light can be obtained, including certain specific colors. When the amniotic fluid is abnormal, e.g. bloody, it will have a red color, so the red wavelength of light will be reflected back more in the reflection measurement and the blood in the fluid will be detected. Since the colors of the amniotic fluid start with light green and go to brown and black, the color of meconium will be detected by using an oem spectrometer that can work more sensitively in this region.

Due to the fact that the invention only works on the basis of color reflectance measurement;
An advantage of the invention is that the screening procedure can be performed without any invasive procedures on the pregnant woman and the fetus. The invention is non-invasive.
An important advantage is that it does not require sampling of amniotic fluid.
Since it is based on reflection measurement, there is no harm to the fetus.
The evaluation result will be immediately visible on the device's screen at the moment of application.

It will also enable screening in the early stages of the birth process. The device is portable, portable and easy to use. It does not need to be connected to a large device such as a computer. It can operate with battery.

The innovation is that a source with a broad spectrum between 400 nm and 1100 nm will be used as a light source, and an OEM spectrometer with a CCD detector or PDA photodiode array will be used as a detector. We are also based on “color measurement” color measurement, reflection. We will not examine any fetal or maternal tissues.

### REFERENCE LIST

1. Light source
2. Fiber bundle carrying the light sent to the reflection probe
3. Reflection probe
4. Fiber carrying the reflected light to the reflection probe
5. CCD detector
6. Spectrometer
7. Monitor
8. Computer
9. Microcontroller

### BRIEF DESCRIPTION OF DRAWINGS

FIG. 1: Representative general view of the inventive reflection probe

FIG. 1.1.Cross-sectional view of the fiber bundle carrying the light sent to the reflection probe

FIG. 1.2. Cross-sectional view of the fiber carrying the reflected light to the reflection probe

FIG. 1.3. Reflection probe fiber section representation view where the transmitted light and the reflected light are combined.

FIG. 2: Representative general device and operating principle view optimizing said invention

FIG. 3: Representative view of the inventive prototype

### DESCRIPTION OF THE INVENTION

Elements of said invention is as follows;

Light source (1), which refers to RGB LED or Tungsten halogen lamp.

The fiber bundle (2), which is the component of the reflection probe (3) and consists of 6 fibers, carries the light from the light source (1) to the reflection probe (3), which carries the transmitted light.

A Reflection probe (3) is a combination of 6 fibers carrying the light from the light source (1) and 1 fiber carrying the reflected light to the spectrometer (6). It looks like the letter “Y”. One of the arms of the letter is connected to the light source (1) and the other to the spectrometer (6). The lower end where the fibers join is held to the measurement location.

The fiber (4), which is a component of the reflection probe (3), which consists of 1 fiber and carries the reflected light to the reflection probe (6), where the reflected light is carried to the spectrometer (6).

A spectrometer (6) is a device that contains a monochromator, a CCD detector (5) or a photodiode array detector (PDA) and an electronic board for sending the collected light data to a computer (8), which contains all these components for detecting light.

Monitor (7) used to visually present the collected data.

Computer (8) used in prototype development. (Mini developer cards will be used instead.)

Microcontroller (9), which refers to mini developer cards or mini-computers used to process the data from the spectrometer (6) and display meaningful data on the monitor (7) or to collect the data.

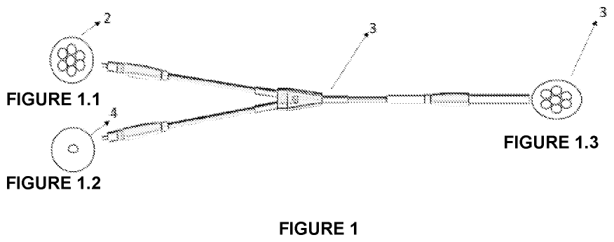

Spectroscopic reflection measurements are made with a fiber bundle called “Reflection Probe” (3). The reflection probe has 7 fibers and the total diameter of the fiber bundle does not exceed a few millimeters (about 5 mm, depending on the intended use, it can vary up to 12 mm). Of these 7 fibers, the fiber to the spectrometer (6) is located in the center and surrounded by the fiber bundle (2) that carries the light sent to the other 6 reflection probes (FIG. 1). The light sent from the light source (1) is carried by the fiber bundle (2) that carries the light sent to these 6 reflection probes and the light is projected onto the sample to be reflected. The light reflected from the sample is collected by the fiber (4), which carries the reflected light to the reflection probe in the center, and transmitted to the Spectrometer (6).

Reflection measurement starts with dark measurement. In this section, no light is allowed to enter the Spectrometer (6). A reference measurement is then taken from the fiber bundle (2) (6 pieces) that carries the light sent to the reflection probe, from a (white) sample from which the full spectrum of the transmitted light can be reflected back. After these two measurements are completed, the reflection of the prepared sample is measured. The wavelength of the reflected light, for example, is intensified depending on its color. Components at the same wavelength as the sample are intensified while other components are absorbed by the sample. This provides information about the color of the sample. There are many applications in the literature based on reflectance measurement. These applications include measuring the thickness of metallic, semiconductor and dielectric layers, fresh and processed product analysis in the food industry, and sensor applications.

RGB led and tungsten halogen lamp will be used as light source (1) in the product. While tungsten halogen gives light in all visible region wavelengths, with RGB led, all visible region light can be obtained, including certain specific colors. When the amniotic fluid is abnormal, e.g. bloody, it will have a red color, so the red wavelength of light will be reflected back more in the reflection measurement and the blood in the fluid will be detected. Since the colors of the amniotic fluid start with light green and go to brown and black, the color of meconium will be detected by using an oem spectrometer (6) that can work more sensitively in this region.

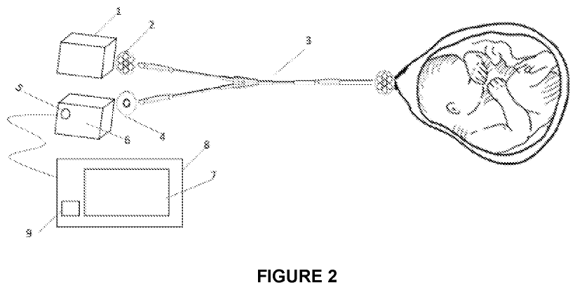

The reflection probe (3) is moved as close as possible to the embryo sac. A beam of light with the optimum pulse width is then sent into the sac. Some of the light beam that enters through the sac reflects off the sac and some reflects off the amniotic fluid inside. The light beam reflected on the reflection probe is collected by the reflection probe (3) and sent to the Spectrometer (6). FIG. 2 shows a visualization of the method.

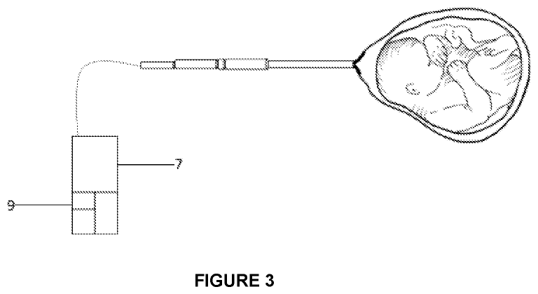

Since the coloration in amniotic fluid is in the visible region, initial studies will be performed using a spectrometer (6) that works in the visible region (specifically between 400 nm and 1100 nm). The information from the spectrometer (6) will be transferred to the computer (8) and the collected data will be analyzed. The position of the reflection probe (3) to the sac will optimize the intensity and pulse duration of the transmitted light. A programmable microcontroller (9) and computer (8) unit will then be deactivated and made into a more miniature prototype. When prototyping the product, an OEM spectrometer (6) will be used as the spectrometer and a high power RGB LED will be used as the light source (1). The colors that the possible meconium fluid can take will be produced with RGB LED and the reflected light will be analyzed so as to increase the accuracy of the measurement. FIG. 3 shows the prototype to be developed.

Preliminary studies were carried out for the project and reflection measurements were made on amniotic fluid samples. In preliminary studies, liquids were placed in plastic and transparent sample containers. While in normal amniotic fluid all of the transmitted light was recovered, red and brownish colors could be detected in bloody and meconium amniotic fluid, respectively.

The small white circle in the color diagram is the color determined after the reference measurement. The LS1 product of Ocean Optic was used as light source (1). The light source contains all wavelengths but has a yellowish appearance (gives the same spectrum as the sun). Therefore, after the reference measurement, the circle in the center of the diagram, which was expected to be in white, appeared in the yellow region. The other black circle in the color diagram gives us information about the color of the amniotic fluid after reflection. For example, if the white circle and the black circle are almost on top of each other in the color diagram, we can say that the liquid reflects all the transmitted light at the same rate. For example; in bloody fluid, the black circle has moved away from the reference (white) circle on a line that turns towards red. In another measurement, the black circle has moved away from the reference circle on a straight line towards a slightly brownish region between yellow and orange (since the relevant measurements are in color, they are not shared to avoid exceeding the drawing rules and are only described in text).

This invention is a non-invasive meconium and blood screening device in amniotic fluid during labor, characterized in that, it comprises of the following; a high-power RGB LED light source (1), a reflection probe (3), where the transmitted light and the reflected light-carrying fibers are combined, an oem spectrometer (6), a monitor (7) on which the analysis of reflected light is displayed, and a programmable microcontroller (9).

This invention, which is called non-invasive meconium and blood screening device in amniotic fluid during the birth process; consists of a programmable microcontroller (9), spectrometer (6), reflection probe (3) and a monitor (7) on which the results will be displayed. A process based on the reflection of the light sent from the light source to the reflection probe (3), which can be positioned in the amniotic sac at different angles, from the amniotic fluid, characterized in that, it comprises the following process steps;
carrying the light sent from the light source (1) by the fiber bundle (2) consisting of 6 fibers, which is the light carrying arm of the reflection probe, and reflecting of light on the sample to be measured,
collecting the light reflected from the sample with the fiber (4) that carries the light reflected to the reflection probe in the center and transmitting the same to the spectrometer (6),
starting the reflection measurement with dark measurement,
taking a reference measurement from the fiber bundle (2) (6 pieces) that carries the light sent to the reflection probe, from a (white) sample from which the full spectrum of the transmitted light can be reflected back,
after these two measurements are completed, taking a reflection measurement from the prepared sample (where the measurement will be taken from the amniotic sac),
moving the reflection probe (3) as close as possible to the embryo sac,
then, sending a beam of light with the optimum pulse width into the sac. Some of the light beam that enters through the sac reflecting off the sac and some reflecting off the amniotic fluid inside,
collecting the light beam reflected on the reflection probe by the reflection probe (3) and sending the same to the Spectrometer (6),
since the coloration in amniotic fluid is in the visible region, using a spectrometer (6) operating in the visible region (especially between 400 nm and 1100 nm) in the first studies, transferring the information received from the spectrometer (6) to the computer (8) and analyzing the collected data,
optimizing the position of the reflection probe (3) to the sac, the intensity of the transmitted light and the duration of the pulse.

1. A process which consists of a programmable microcontroller (9), spectrometer (6), reflection probe (3) and a monitor (7) on which the results will be displayed, relies on the reflection of the light sent from the light source (1) back from the amniotic fluid to the reflection probe (3), which can be positioned in the amniotic sac at different angles of the non-invasive meconium and blood scanning device in the amniotic fluid during the birth process, characterized in that, it comprises the following process steps;
carrying the light sent from the light source (1) by the fiber bundle (2) consisting of 6 fibers, which is the light carrying arm of the reflection probe (3), and reflecting of light on the sample to be measured,
collecting the light reflected from the sample with the fiber (4) that carries the light reflected to the reflection probe in the center and transmitting the same to the spectrometer (6),
starting the reflection measurement with dark measurement,
taking a reference measurement from the fiber bundle (2) (6 pieces) that carries the light sent to the reflection probe (3), from a (white) sample from which the full spectrum of the transmitted light can be reflected back,
after these two measurements are completed, taking a reflection measurement from the prepared sample (where the measurement will be taken from the amniotic sac),
Moving the reflection probe (3) as close as possible to the embryo sac,
sending a beam of light with the optimum pulse width is then sent into the sac. Some of the light beam that enters through the sac reflecting off the sac and some reflecting off the amniotic fluid inside,
collecting the light beam reflected on the reflection probe by the reflection probe (3) and sending the same to the Spectrometer (6),
since the coloration in amniotic fluid is in the visible region, using a spectrometer (6) operating in the visible region (especially between 400 nm and 1100 nm) in the first studies, transferring the information received from the spectrometer (6) to the computer (8) and analyzing the collected data,
optimizing the position of the reflection probe (3) to the sac, the intensity of the transmitted light and the duration of the pulse.

2. A non-invasive meconium and blood screening device in amniotic fluid during the birth process, characterized in that, it comprises of the following; a high-power RGB LED light source (1), a reflection probe (3), where the transmitted light and the reflected light-carrying fibers are combined, an oem spectrometer (6), a monitor (7) on which the analysis of reflected light is displayed, and a programmable microcontroller (9).

3. A non-invasive meconium and blood screening device in amniotic fluid during the birth process according to claim 2, characterized in that; it comprises a fiber bundle (2), which is the component of the reflection probe (3) and consists of 6 fibers, where the light from the light source (1) is carried and the light sent to the reflection probe is carried.

4. A non-invasive meconium and blood screening device in amniotic fluid during the birth process according to claim 2, characterized in that; it comprises fiber (4), which is a component of the reflection probe (3) and consists of 1 fiber, and the reflected light is carried to the reflection probe, where the reflected light is carried to the spectrometer (6).
